(19) United States
(12) Patent Application Publication
(10) Pub. No.: US 2025/0082519 A1
(43) Pub. Date: Mar. 13, 2025

(54) SANITARY NAPKIN

(71) Applicant: Jenny Vallance, Nanaimo (CA)

(72) Inventor: Jenny Vallance, Nanaimo (CA)

(21) Appl. No.: 18/464,478

(22) Filed: Sep. 11, 2023

(51) Int. Cl. A61F 13/56; A61F 13/472; A61F 13/505

(52) U.S. Cl. CPC A61F 13/5605; A61F 13/472; A61F 13/505

(57) ABSTRACT

A sanitary napkin configured to be reusable wherein the sanitary napkin of the present invention is releasably secure to a user's legs. The invention includes a liner member and a pad member wherein the pad member is configured to be able to be washed and reused. The pad member of the invention is manufactured from three layers with one layer being a super absorbent material. The pad member includes a keeper configured to releasably secure the pad member to the liner member. The liner member is mateably shaped to the pad member and includes passages on the opposing longitudinal edges thereof. Secured to the liner member are a first securing member and a second securing member wherein a portion of each are journaled through the passages on the opposing longitudinal edges. The first securing member and second securing member are adjustable in length and surroundably mount legs of a user.

### FIELD OF THE INVENTION

The present invention relates generally to feminine hygiene products, more specifically but not by way of limitation, reusable sanitary napkin wherein the present invention includes a liner member wherein the liner member has releasably secured thereto an absorbent pad that is reusable and wherein the liner member includes a first securing member and a second securing member on opposing sides thereof.

### BACKGROUND

Sanitary napkins can be utilized by women to help with bladder incontinence and keep fluids such as urine from egressing onto clothes and the like. Conventional sanitary napkins and adult diaper style devices are commercially available in various sizes and styles. The commercially available styles perform the same basic task and choice thereof is made based on a variety of personal reasons.

One issue with existing sanitary napkins and bladder control products is the amount of plastic utilized. As millions of sanitary napkins and adult diapers are utilized every day the plastic is introduced into waste water and landfills which has a negative impact on the environment. Additionally, commercially available diapers and sanitary napkins are quite expensive and those with lower incomes can have hardships having to purchase the sanitary napkins. Lastly, commercially available bladder control products and sanitary napkins are not reusable and as such the economics thereof are less than optimal.

Accordingly, there is a need for a sanitary napkin that is reusable and washable wherein the sanitary napkin of the present invention is operable to reduce impact on the environment and provide a comfortable and convenient technique to capture bodily fluids.

### SUMMARY OF THE INVENTION

It is the object of the present invention to provide a reusable sanitary napkin operable to manage bodily fluids wherein the present invention includes a liner member wherein the liner member is rectangular in shape.

Another object of the present invention is to provide a sanitary napkin that is configured to be washable that further includes a pad member that can be cleaned and reused wherein the pad member is releasably secured to the liner member.

A further object of the present invention is to provide a reusable sanitary napkin operable to manage bodily fluids wherein the liner member includes opposing longitudinal edges wherein the opposing longitudinal edges have passages formed therein.

Yet a further object of the present invention is to provide a sanitary napkin that is configured to be washable wherein the liner member includes a first securing member and a second securing member secure to the opposing longitudinal edges.

Still another object of the present invention is to provide a reusable sanitary napkin operable to manage bodily fluids wherein a portion of the first securing member and second securing member are journaled through the passages formed on the longitudinal edges of the liner member.

An additional object of the present invention is to provide a sanitary napkin that is configured to be washable wherein the pad member is manufactured from three layer wherein the middle layer is manufactured from a super absorbent material.

Yet a further object of the present invention is to provide a reusable sanitary napkin operable to manage bodily fluids wherein the first securing member and second securing member are configured to be surroundably mounted the legs of a user.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a front view of the liner member of the present invention; and

FIG. 2 is a front view of the pad member of the present invention; and

FIG. 3 is a rear view of the liner member of the present invention.

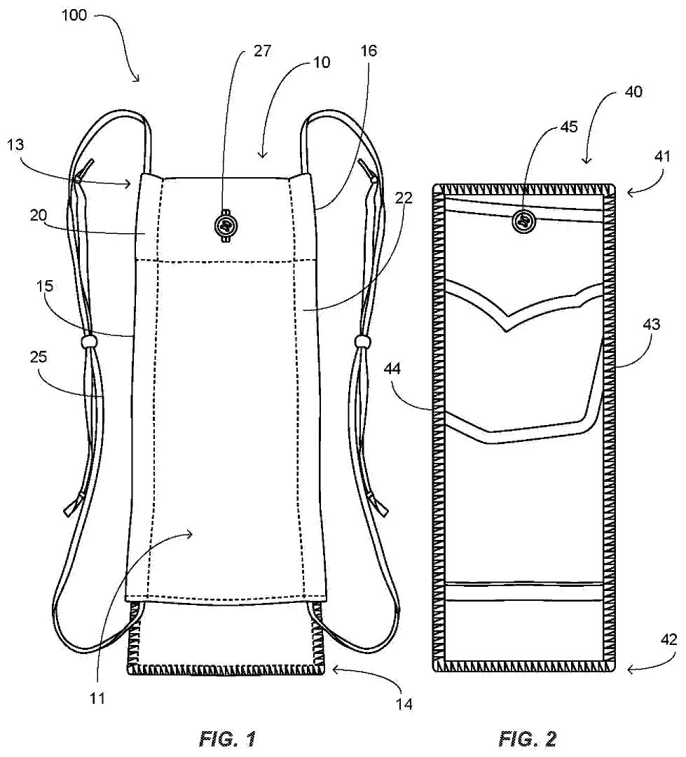

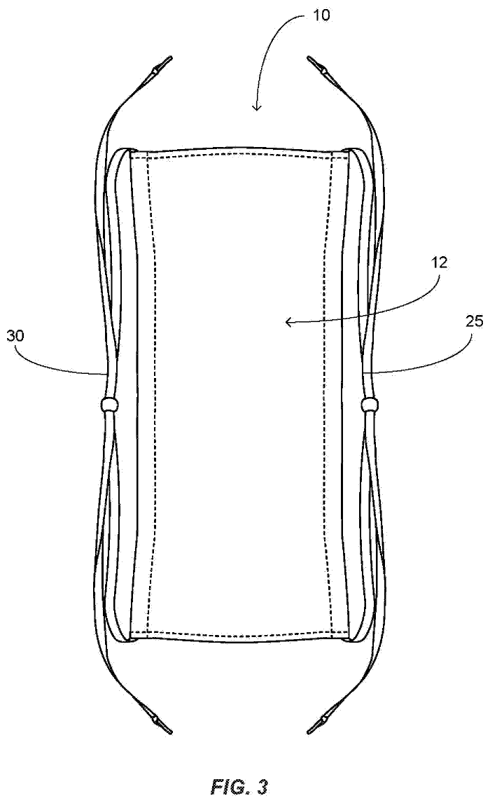

### DETAILED DESCRIPTION

Referring now to the drawings submitted herewith, wherein various elements depicted therein are not necessarily drawn to scale and wherein through the views and figures like elements are referenced with identical reference numerals, there is illustrated a sanitary napkin 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted herewith, the sanitary napkin 100 includes a liner member 10 and a pad member 40 wherein the pad member 40 is configured to be releasably secured against the liner member 10. The pad member 40 is mateably shaped with the liner member 10 with the preferred embodiment being rectangular in shape. The pad member 40 includes a first end 41, a second end 42 and opposing longitudinal edges 43,44. The pad member 40 further includes a keeper 45 wherein the keeper 45 is configured to releasably secure the pad member 40 to the liner member 10. It should be understood within the scope of the present invention that the keeper 45 could be but is not limited to a button or a snap. The pad member 40 is manufactured from three layers (not particularly illustrated herein). The three layers of the pad member 40 are adjacent with the middle layer being manufactured from a super absorbent material and the layers on each side of the middle layer being manufactured from a fabric. In a preferred embodiment of the sanitary napkin 100, the middle layer is manufactured from a rayon polypropylene blend. It should be understood within the scope of the present invention that the pad member 40 is provided in alternate sizes in order to mateably secure to the liner member 10.

The sanitary napkin 100 includes a liner member 10. The liner member 10 is rectangular in shape having a first side 11 and a second side 12. The liner member 10 includes a first end 13 and second end 14. The liner member 10 includes a first longitudinal edge 15 and second longitudinal edge 16. The first longitudinal edge 15 and second longitudinal edge 16 include first passage 20 and second passage 22 respectively formed therealong utilizing suitable techniques. The first passage 20 and the second passage 22 extend between the first end 13 and second end 14 having openings at both the first end 13 and second end 14.

The sanitary napkin 100 includes a first securing member 25 and a second securing member 30. The first securing member 25 is operably coupled to the first longitudinal edge 15 wherein a portion thereof is journaled through the first passage 20. The second securing member 30 is operably coupled to the second longitudinal edge 16 wherein a portion thereof is journaled through the second passage 22. Both the first securing member 25 and second securing member 30 are adjustable in length and are configured to be surroundably mounted to a user's legs. In a preferred embodiment of the present invention, the first securing member 25 and second securing member 30 are manufactured from elastic bands. The liner member 10 further includes keeper 27 wherein the keeper 27 is configured to releasably secure to keeper 45.

The liner member 10 is manufactured from three layers (not particularly illustrated herein). In a preferred embodiment of the liner member 10, the liner member 10 includes a middle layer manufactured from plastic and layers on opposing sides of the middle layer manufactured from fabric. It should be understood within the scope of the present invention that various types of fabric could be utilized.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A sanitary napkin operable to be cleaned and reused wherein the sanitary napkin comprises:
a liner member, said liner member being rectangular in shape, said pad member having a first side and a second side, said liner member having a first end and a second end, said liner member having a first longitudinal edge and a second longitudinal edge, said liner member having a first passage, said first passage being formed along said first longitudinal edge, said first passage extending intermediate said first end and said second end of said liner member, said liner member having a second passage, said second passage being formed along said second longitudinal edge, said second passage extending intermediate said first end and said second end of said liner member;
a pad member, said pad member being rectangular in shape, said pad member being manufactured from three layers, said three layers having a middle layer, said middle layer being manufactured from an absorbent material, said pad member configured to be releasably secured to said second side of said liner member;
a first securing member, said first securing member being operably coupled to said first longitudinal edge of said liner member, said first securing member having a portion thereof being journaled through said first passage;
a second securing member, said second securing member being operably coupled to said second longitudinal edge of said liner member, said second securing member having a portion thereof being journaled through said second passage.

2. The sanitary napkin operable to be cleaned and reused as recited in claim 1, wherein said pad member further includes a keeper, said keeper of said pad member operable to releasably secure said pad member to said liner member.

3. The sanitary napkin operable to be cleaned and reused as recited in claim 2, wherein said liner member further includes a fastener, said fastener configured to operably couple with said keeper.

4. The sanitary napkin operable to be cleaned and reused as recited in claim 3, wherein said middle layer of said pad member is manufactured from a rayon and polypropylene blend.

5. The sanitary napkin operable to be cleaned and reused as recited in claim 4, wherein said first securing member is an elastic band.

6. The sanitary napkin operable to be cleaned and reused as recited in claim 5, wherein said second securing member is an elastic band.

7. The sanitary napkin operable to be cleaned and reused as recited in claim 6, wherein said first securing member and said second securing member are adjustable in length.

8. The sanitary napkin operable to be cleaned and reused as recited in claim 7, wherein said liner member is manufactured from three layers wherein a middle layer of said liner member is manufactured from plastic.
